(19) United States
(12) Patent Application Publication
(10) Pub. No.: US 2020/0283824 A1
(43) Pub. Date: Sep. 10, 2020

(54) USE OF MEGF11 AS DIAGNOSTIC AND PROGNOSTIC BIOMARKER AND THERAPEUTIC TARGET FOR TRIPLE NEGATIVE BREAST CANCER

(71) Applicant: Taipei Veterans General Hospital, Taipei City (TW)

(72) Inventors: Ling-Ming TSENG, Taipei (TW); Jen-Hwey CHIU, Taipei (TW); Chun-Yu LIU, Taipei (TW); Jir-You WANG, Taipei (TW)

(21) Appl. No.: 16/811,125

(22) Filed: Mar. 6, 2020

Related U.S. Application Data
(60) Provisional application No. 62/814,483, filed on Mar. 6, 2019.

(51) Int. Cl. C12Q 1/686; A61K 31/7105

(52) U.S. Cl. CPC C12Q 1/686; A61K 31/7105; C12Q 2561/113; G01N 2800/54; G01N 2333/485

(57) ABSTRACT

The invention discloses a method for diagnosing recurrence and treating a triple negative breast cancer (TNBC) in a subject by use of multiple epidermal growth factor-like domains 11 (MEGF11) as a diagnostic and prognostic biomarker and a therapeutic target.

### CROSS-REFERENCE TO RELATED APPLICATION

This utility application claims priorities to U.S. Provisional Application Ser. No. 62/814,483, filed Mar. 6, 2019, which is incorporated herein by reference.

### BACKGROUND OF THE INVENTION

### 1. Field of the Invention

The invention relates to a method for diagnosing recurrence and treating a triple negative breast cancer (TNBC) in a subject, and more in particular to a method or diagnosing recurrence and treating a TNBC in a subject by use of multiple epidermal growth factor-like domains 11 (MEGF11) as a diagnostic and prognostic biomarker and a therapeutic target.

### 2. Description of the Prior Art

Breast cancer is the most common invasive female cancer worldwide [1, 2]. Triple negative breast cancer (TNBC) is characterized by occurrence in younger women, aggressive tumor behavior, and a high association with metastasis to distant organs. TNBC has strong resistance to hormonal therapy, chemotherapy, and target therapy [3-5]. Many biomarkers associated specifically with TNBC subtypes have been identified [6-9] and several target therapies such as receptor tyrosine kinase (RTK) and Src family inhibitors have been investigated in clinical trials [10]. Nevertheless, the results seem to be of limit usefulness in TNBC patients.

Epidermal growth factor (EGF)-like domain, a highly conserved protein domain, has been found in a large number of animal proteins [11]. Based on different functions involved in multiple EGF like domains, distinct domain subtypes have been identified [12]. Previous investigations have shown that EGF-like domains play important roles in immune responses [13], apoptosis [14], and calcium binding [15, 16]. Recently, much effort has been devoted to studies of correlation between MEGF subtypes and their functions. For examples, MEGF10 is postulated as a tumor repressor gene in neuroblastoma [17], while genetic aberrance of MEGF10 causes myopathy [18, 19], areflexia, respiratory distress and dysphagia (EMARDD) [20]. Mutation in MEGF8 is highly associated with Carpenter syndrome subtype with defective left-right patterning [21]. Nonetheless, the functions of many MEGF subtypes, such as MEGF6, MEGF7, MEGF9, and MEGF11, remains further elucidated.

Up to date, few reports mention the role of MEGF11 in mammalian species, although it shares a substantial homology with MEGF10 and they are likely to represent a novel protein family [22, 23]. Recent evidence has demonstrated that MEGF10 and MEGF11 play a crucial role in the formation of mosaics by two retinal interneuron subtypes, starburst amacrine cells and horizontal cells [24]. However, information concerning the role of MEGF11 on cancer cells, especially triple negative breast cancer (TNBC), is lacking.

Recent bio-information study which targeted the molecular mechanisms in TNBC tumors demonstrated that several genes were differentially expressed in paired recurrent and non-recurrent [25]. In addition, the inventors of this present invention conduct cDNA open array analysis for 224 genes on paired TNBC tissue samples (16 recurrent and 24 non-recurrent tissues) to disclose that MEGF11 was significantly up-regulated in tumor tissues with subsequent clinical recurrence than those without recurrence.

### SUMMARY OF THE INVENTION

Accordingly, one scope of the invention is to elucidate the role of MEGF11 on human TNBC cells, both in vitro, in vivo and in human tissues.

Accordingly, another scope of the invention is to provide a method or diagnosing recurrence and treating a TNBC in a subject by use of MEGF11 as a diagnostic and prognostic biomarker and a therapeutic target.

A method according to the first preferred embodiment of the invention is for diagnosing recurrence and treating a TNBC in a subject. Firstly, the method according to the first preferred embodiment of the invention is to obtain a first sample from the subject and a second sample from a control culture. Then, the method according to the first preferred embodiment of the invention is to identify a first relative protein amount of MEGF11 in the first sample and a second relative protein amount of MEGF11 in the second sample by use of using a PCR-based way. Next, the method according to the first preferred embodiment of the invention is to compare the first relative protein amount with the second relative protein amount. Subsequently, if the comparing result in the aforesaid step indicates that the treated subject has an expression of MEGF11 greater than that of the control culture, the method according to the first preferred embodiment of the invention is to diagnose the subject as being in a risk of recurrence of the TNBC. Finally, the method according to the first preferred embodiment of the invention is to administer the subject an effective amount of a composition including an shRNA that knocks down MEGF11 expression.

A method according to the second preferred embodiment of the invention is for diagnosing recurrence and treating a TNBC in a subject. Firstly, the method according to the second preferred embodiment of the invention is to obtain a sample from the subject. Then, the method according to the second preferred embodiment of the invention is to identify a protein expression of MEGF11 in the sample by use of using a PCR-based way. Next, the method according to the second preferred embodiment of the invention is to semi-quantify the protein expression of MEGF11 in the sample. Subsequently, the method according to the second preferred embodiment of the invention is to express the semi-quantified the protein expression of MEGF11 in the sample as being a determined value. Afterward, the method according to the second preferred embodiment of the invention is to judge if the determined value is equal to or greater than a threshold. Then, if the judging result in the aforesaid step is YES, the method according to the second preferred embodiment of the invention is to diagnose the subject as being in a risk of recurrence of the TNBC. Finally, the method according to the second preferred embodiment of the invention is to administer the subject an effective amount of a composition including a shRNA that knocks down MEGF11 expression.

In one embodiment, the PCR-based way includes one or more of a RT-PCR and a real-time PCR.

The advantage and spirit of the invention may be understood by the following recitations together with the appended drawings.

### BRIEF DESCRIPTION OF THE APPENDED DRAWINGS

FIGS. 1a to 1d show identification of MEGF11 in recurrent triple negative breast cancer. Using cDNA open array chips, 224 genes in paired TNBC tissue samples (16 recurrent and 24 non-recurrent tissues) was analyzed and MEGF11 was significantly up-regulated in tumor tissues with subsequent clinical recurrence than those without recurrence (FIG. 1a). Protein expression by immunohistochemistry (FIG. 1b) was correlated with patients' survival such as recurrence-free survival (FIG. 1c) and overall survival (FIG. 1d). The protein expression of MEGF11 was semi-quantified and expressed as (0), <10%, (1), 11-25%, (2), 26-50%, (3) >50% of tumor cells. Asterisk indicated a p value <0.05 by Mann-Whitney U test and Kaplan-Meier survival analysis was performed with Prism 5 software.

FIGS. 2a to 2g show that knocked down MEGF11 in TNBC cell lines decreases cell proliferation through suppression of AKT, mTOR and NF-κB signaling pathways. MEGF11 was knocked down with short hairpin RNA (shRNA) in TNBC cell lines MDA-MB-231 and MDA-MB-468 cells. Cell proliferation-related signaling such as AKT, ERK (FIG. 2a), mTOR, and NF-κB (FIG. 2b) and nuclear factors NF-κB p65, CREB, and AP-1 (FIG. 2c) were analyzed with Western blot (n=4-6). Cell migration activity (FIG. 2d) and in vivo tumor growth rate (FIG. 2e) were evaluated by wound healing assay (n=6) and in vivo imaging system (IVIS) in nude mice (n=6), respectively. The mRNA transcripts of chemokines such as CCL20, CXCL2, CXCL5, and CXCL11 (FIG. 2f), and cytokines such as IL1β, TNF-α, IL6, and IL8 (FIG. 2g) were quantified with real-time PCR (n=4-6). Asterisks indicate a p value <0.05 in ΔMEGF11 TNBC cells compared to the wild type by Mann-Whitney U test.

FIGS. 3a to 3f shows that over-expression of MEGF11 does not promoter cell proliferation. The MEGF11 over-expression vector was cloned into the pCMV-AC-GFP vector. After MEGF11 was over-expressed in TNBC cells, the cell number (FIG. 3a) were evaluated by Trypan blue exclusion assay and verified by cell cycle analysis (FIG. 3b). The growth related signaling such as AKT, ERK, mTOR, and p70s6K (c, d), and nuclear factors NF-κ B, CREB, and AP-1 (FIGS. 3e and 3f) were analyzed with Western blot and quantified using wild type as control group. The growth curves were analyzed with Two-way ANOVA. Asterisks indicate a p value <0.05 in o/e MEGF11 TNBC cells compared to the scramble group by Mann-Whitney U test (n=4).

FIGS. 4a to 4f show that over-expression of MEGF11 increases up-regulation of chemokines, proinflammatory cytokines gene expression. Following ingenuity pathway analysis (FIG. 4a), chemokines such as CCL20, CXCL2, CXCL5 and IL-17A expression in o/e MEGF11 MDA-MB-231 line (FIGS. 4b and 4c) and MDA-MB-468 cells (FIG. 4d) were analyzed with Western blot (n=5) and quantified using wild type as control group. The mRNA transcripts of chemokines such as CCL20, CXCL2, CXCL5 (FIG. 4e), and cytokines such as TNF-α, IL1β, IL-6, IL-8 and COX2 (f) were quantified with real-time PCR. Asterisks indicate a p value <0.05 in o/e MEGF11 TNBC cells compared to the scramble group by Mann-Whitney U or student t test (n=3-4).

FIGS. 5a to 5f show cross talk between MEGF11 and IL-17A. MEGF11 was knocked down with short hairpin RNA (shRNA) and over-expressed with pCMV-AC-GFP vector in TNBC cell lines MDA-MB-231 and MDA-MB-468 cells. When MEGF11 gene was knocked down, mRNA transcripts of IL-17A in ΔMEGF11 (FIG. 5a) and o/e MEGF11 (FIG. 5b) were quantified by real-time PCR (n=4), respectively. After administration of different doses (0-, 0.001, 0.1 ng/mL) of IL-17A in cultured media, MEGF11 protein (FIG. 5c) and IL-17A-related signaling protein in MDA-MB-231 (FIG. 5d) and MDA-MB-468 (FIG. 5e) were analyzed with Western blot (n=4-6). The mRNA transcripts of MEGF11, IL-17A, IL-17 receptors (IL17RB, IL17RC) in MDA-MB-231 and MDA-MB-468 cells (FIG. 5f) were quantified with real-time PCR (n=6), respectively. Data between two groups were analyzed with Mann-Whitney U or student t test, while dose-related data were analyzed with one way ANOVA, followed by Dunnet's post hoc test. Asterisks indicate a p value <0.05 compared to the wild type (for ΔMEGF11) or scramble group (for o/e MEGF11) or the vehicle group (for dose-dependent study).

FIGS. 6a to 6g show that IL-17A increases up-regulation of chemokines, proinflammatory cytokines gene expression. After administration of different doses (0-, 0.001, 0.1 ng/mL) of IL-17A in cultured media of MDA-MB-231 and MDA-MB-468 cells, chemokines such as CCL20, CXCL2, and CXCL5 (FIGS. 6a, 6b and 6c) were analyzed with Western blot (n=4). The mRNA transcripts of chemokines (FIGS. 6d and 6 e) and pro-inflammatory cytokines such as TNF α, IL-1β, IL-6, IL-8, and COX2 (FIGS. 6f and 6g) were quantified with real-time PCR (n=6), respectively. Data were analyzed with one way ANOVA, followed by Dunnet's post hoc test. Asterisks indicate a p value <0.05 compared to the vehicle group.

FIGS. 7a to 7f show that knocked down MEGF11 in mouse 4T1 mammary cancer cell line decreases tumor weight and circulating tumor cells. Using spontaneously occurred mouse mammary tumor 4T1 cell line as in vivo metastatic model, 4T1 cells (1×10⁷ cells/0.1 ml PBS) of MEGF11 wild type (n=9) and Δ MEGF11 4T1 (n=8) were orthotopically injected into two fat pads (left upper and right lower mammary glands). After two weeks, the 4T1 bearing mice were anesthetized, the blood cells were collected and centrifuged with Ficoll-Paque (density: 1.084). The tumor weight (FIG. 7a) was measured. The implanted tumors were homogenized and analyzed with Western blot (FIGS. 7b and 7c). The peripheral mononuclear cells were harvested for primary cultures and circulating 4T1 cells were selected with 6-thioguanine (60 μM) (FIG. 7d) which was analyzed by Fisher's exact test, followed by quantification by agar assay (FIGS. 7e and 7f). A p value <0.05 indicates statistical significance in ΔMEGF11 4T1 cells compared to the wild type by Mann-Whitney U test (*) or by student t test (#). NC, negative control; PC, positive control.

### DETAILED DESCRIPTION OF THE INVENTION

Triple negative breast cancer (TNBC) is characterized by its high metastasis and recurrence rate. The inventors' previous study demonstrated that up-regulated multiple epidermal growth factor-like domains 11 (MEGF11) gene expression was involved the recurrence mechanism of triple negative breast cancer. Accordingly, the aim of the invention is to elucidate the role of MEGF11 expression in TNBC cells, both in vitro and in vivo and in human tissues.

Using human tumor tissue array, the expression of MEGF11 is correlated with patients' prognosis, including recurrence-free and overall survival. MEGF11 gene is knocked down or over-expressed in MDA-MB-231/468 cells and gene expression of cell growth and chemokines are evaluated by Western blot and real-time PCR. Tumor growth of implanted human TNBC cells and circulating tumor cells using mouse beast tumor 4T1 cells are used for in vivo studies.

After MEGF11 knocked down, there is a significant decreased cell growth via inhibition of AKT, NF-κB, CREB and AP-1 activation in both MDA-MB-231/468 cells, and suppressed tumor growth and decreased mouse circulating 4T1 breast cancer cells in vivo. Surprisingly, over-expression MEGF11 increased up-regulation of chemokines (CCL20, CXCL2, etc.), proinflammatory cytokines gene expression via AKT activation, but not increased cell proliferation. MEGF11 is shown positively cross-talked with IL-17A signaling. Furthermore, patients with over-expressed MEGF11 tumors had poor prognosis in recurrence-free and overall survival clinically.

The inventors' novel findings demonstrates that MEGF11 is essential for tumor survival and overexpressed MEGF11 induced cytokines and chemokines cascades, which favored tumor microenvironment for distant metastasis. MEGF11 can be a potential therapeutic target for preventing TNBC recurrence.

Accordingly, one scope of the invention is to provide a method or diagnosing recurrence and treating a TNBC in a subject by use of MEGF11 as a diagnostic and prognostic biomarker and a therapeutic target.

A method according to the first preferred embodiment of the invention is for diagnosing recurrence and treating a TNBC in a subject. Firstly, the method according to the first preferred embodiment of the invention is to obtain a first sample from the subject and a second sample from a control culture. Then, the method according to the first preferred embodiment of the invention is to identify a first relative protein amount of MEGF11 in the first sample and a second relative protein amount of MEGF11 in the second sample by use of using a PCR-based way. Next, the method according to the first preferred embodiment of the invention is to compare the first relative protein amount with the second relative protein amount. Subsequently, if the comparing result in the aforesaid step indicates that the treated subject has an expression of MEGF11 greater than that of the control culture, the method according to the first preferred embodiment of the invention is to diagnose the subject as being in a risk of recurrence of the TNBC. Finally, the method according to the first preferred embodiment of the invention is to administer the subject an effective amount of a composition including a shRNA that knocks down MEGF11 expression.

In one embodiment, the control culture includes non-recurrent tissues, non-recurrent cells and non-recurrent bloods of the TNBC.

In one embodiment, the PCR-based way includes one or more of a RT-PCR and a real-time PCR.

A method according to the second preferred embodiment of the invention is for diagnosing recurrence and treating a TNBC in a subject. Firstly, the method according to the second preferred embodiment of the invention is to obtain a sample from the subject. Then, the method according to the second preferred embodiment of the invention is to identify a protein expression of MEGF11 in the sample by use of using a PCR-based way. Next, the method according to the second preferred embodiment of the invention is to semi-quantify the protein expression of MEGF11 in the sample. Subsequently, the method according to the second preferred embodiment of the invention is to express the semi-quantified the protein expression of MEGF11 in the sample as being a determined value. Afterward, the method according to the second preferred embodiment of the invention is to judge if the determined value is equal to or greater than a threshold. Then, if the judging result in the aforesaid step is YES, the method according to the second preferred embodiment of the invention is to diagnose the subject as being in a risk of recurrence of the TNBC. Finally, the method according to the second preferred embodiment of the invention is to administer the subject an effective amount of a composition including a shRNA that knocks down MEGF11 expression.

In one embodiment, the PCR-based way includes one or more of a RT-PCR and a real-time PCR.

In one embodiment, the threshold is in a range of from 50% to 60%.

This present invention is further illustrated by the following non-limiting examples.

### Examples

Experimental Procedures

Subjects

Human study for tumor tissue utilization from bio-bank was approved by the Institutional Review Board of Taipei Veterans General Hospital (#2013-10-020BC).

Breast cancer patients from January 2001 to December 2010 were diagnosed under tissue proof in our hospital. One hundred and thirty five patients' records such as receptors status of estrogen receptor (ER), progestin receptor (PR), HER2 and clinical outcomes including overall survival (OS) and recurrence-free survival (RSF) were retrospectively reviewed from the database in this hospital. All data were collected during clinical care without direct contact with patients for data collection and analysis and such written consents from study subjects were waived by the institutional review board. The mean follow up time was >5 years. ER or PR >1% was defined as positive while ER or PR<1% as negative.

Immunohistochemistry for MEGF11 Expression

The protein expression of MEGF11 on tissue array (135 tumor samples) in the archives of the department of pathology were performed by immunohistochemical stains for MEGF11 (genetex, GTX120233) which were validated by an expert of pathology. The protein expression of MEGF11 was semi-quantified and expressed as (0), <10%, (1), 11-25%, (2), 26-50%, (3) >50% of tumor cells examined.

Cell Line and Reagents

Human triple negative breast cancer cell line MDA-MB-231 and MDA-MB-468 (ER-, HER2 low) were maintained in F12 MEM (NO. 12400-024, Gibco, NY, USA). Mouse mammary tumor 4T1 cell line [26] was cultured in RPMI medium. They were obtained from American Type Culture Collection (ATCC, Manassas, Va., USA) and supplemented with 10% FBS, 2 mM L-glutamine and penicillin/streptomycin and cultured at 37° C. in a humidified atmosphere containing 5% CO₂. All cell lines were tested as mycoplasma-free.

Short Hairpin RNA (shRNA) Transfection

Short hairpin RNA (shRNA) used to silence MEGF11 gene were obtained from Academia Sinica. One day after MDA-MB-231, MDA-MB-468 or mouse 4T1 cell lines were subcultured, they (30-40% confluent) were transfected for 24 h with shRNA against MEGF11 or non-silencing control using GenePORTER 2 transfection reagent (Genlantis, San Diego, Calif., USA) dissolved in Optimum (Invitrogen) at a final concentration of 80 nM. And then, MDA-MB-231/468 or mouse 4T1 cells were recovered for further experiments. After several passages, the ΔMEGF11 MDA-MB-231/468 and ΔMEGF11 4T1 lines were established by puromycin selection.

Generation of the MEGF11 Expression Vector

The MEGF11 expression vector was generated by amplification of the full-length MEGF11 cDNA from human MDA-MB-231 cells using specific primer pairs (forward primer: 5′-GCGATCGCCATGGTGCTCTCCCTGAC-3; reverse primer: 5′-ACGCGTAGATTGCTTGTCCTGGGACG-3′) and cloned into the pCMV-AC-GFP vector (Origene # PS100010). The construct was verified by DNA sequencing. Then the lentivirus containing o/e MEGF11 construct was made by Academia Sinica, ROC for further studies.

Cell Growth by Trypan Blue Dye Exclusion Assay

MDA-MB-231 and MDA-MB-468 cells were transferred to low serum culture medium with a cell density (1×10⁴/well) in a 12-well plate, followed by treatment of different doses of herbal extracts (0-, 1-, 3 μg/mL). After 1, 2, and 3 days of treatment, cells were washed twice with phosphate-buffered saline (PBS), pH 7.4, and trypsinized using 0.5 mL trypsin-ethylenediamine tetraacetic acid (0.05% trypsin, 0.53 mL ethylenediamine tetraacetic acid I 4Na, Gibco/Invitrogen, New York, N.Y.). Suspended cells were re-suspended in fresh culture medium, followed by counting cell number with hemocytometer-based trypan blue dye exclusion cell quantification.

Cell Migration Assay

In vitro cell migration of MDA-MB-231 cells (MDA-MB-468 is not suitable for this assay) were performed using a cell culture insert[27] (NO. 80209, ibidi, Munich, Germany). In brief, 2×10⁴ cells were seeded within an insert on a 3.5 cm petri dish for overnight, followed by low serum (1% FBS) starvation for 24 h. Following cells washed with PBS, the inserts were removed and the cells were continuously cultured. After 24 h-incubation, migrated cells were examined under a light microscope and photographed. The percentage of migratory cells was calculated compared to negative control.

Western Blotting Analysis

Cultured cells were lysed in a buffer containing 150 mM KCl, 10 mM Tris pH 7.4, 1% Triton X-100, phosphatase inhibitor and protease inhibitors cocktail (Complete Mini; Roche, Mannheim, Germany). The protein concentrations in cell homogenates were measured using Bradford's method [28]. Thirty microgram of proteins were loaded to 10% SDS-PAGE and transferred to a nitrocellulose membrane (Hybond-C; Amersham Biosciences, NJ, USA). The membrane were blocked with 5% bovine serum albumin and probed with specific primary antibodies which were obtained commercially.

Total RNA Extraction and Reverse Transcription-PCR

Total RNA was isolated by using a modified single-step guanidinium thiocyanate method [29] (TRI REAGENT, T-9424, Sigma Chem. Co., St. Louis, Mo., USA). Complementary DNA (cDNA) was prepared from the total RNA complied with the First Strand cDNA Synthesis Kit (Invitrogen, CA, USA). The de novo gene synthesis changed by each treatment group was detected by reverse transcriptase-polymerase chain reaction (RT-PCR). Primers pairs such as MEGF11 (Forward 5′-TGG CTG ACA CTT TCG AAC AC-3′; Reverse 5′-CCT CAT GGA CAT GTT TGC AG-3′) were used commercially available primers. The possible contamination of any PCR component was excluded by performing a PCR reaction with these components in the absence of RT product in each set of experiment (non-template control, NTC). Quantification of RNA transcripts was analyzed according to the method described previously with some modification. For statistical comparison, the relative expression of specific genes mRNA was normalized to the amount of GAPD in the same RNA extracts. All samples were analyzed in triplication.

In Vivo Tumor Xenograft

Study protocols involving experimental mice followed ARRIVE (Animal Research: Reporting of In Vivo Experiments) guidelines and were approved by the Institutional Animal Committee of Yang-Ming University (No. 1050802) and Taipei Veterans General Hospital (No. 2018-029). Immuno-deficient NU-Foxnlnu mice were obtained from National Laboratory Animal Center (Taipei, Taiwan, ROC). They were given ad libitum access to food and water and maintained in a specific pathogen-free environment with 12 hrs. light-dark cycle at 22-24° C. and 50% humidity. The mice were used for experiments at 8 weeks of age. Wild type-, and knocked down MEGF11 (ΔMEGF11) of MDA-MB-231 with luciferase genes cells were injected into back of Immuno-deficient NU-Foxnlnu mice, with a cell density of 1×10⁷ cells/0.1 ml PBS for each mouse, leading to a solid tumor noticeable around the injection site at day 7-14. Then, the progression of tumor size with visualized using an in vivo imaging system (IVIS). For tumor metastasis study, wild type-, and knocked down MEGF11 (ΔMEGF11) mouse mammary 4T1 cells (1×10⁷ cells/0.1 ml PBS) were orthotopically injected into two fat pads (left upper and right lower mammary glands) of 8-wk female BALB/c mice. Then, the mice were sacrificed 8 weeks thereafter or the tumor size was more than 2% of body weight. The tumor size, tumor weight were measured and tumor tissues or suspiciously metastatic organs such as lung and liver were frozen for further analysis.

Selection of Circulating Mouse Mammary Breast Cancer 4T1 Cells

After the 4T1 bearing mice were anesthetized, the blood cells were collected and centrifuged (400 g) with Ficoll-Paque PREMIUM (density: 1.084) (17-5446-02, GE Healthcare Bio-Sciences, Sweden). The peripheral mononuclear cells were primarily cultured for several passages and circulating 4T1 cells were selected with 6-thioguanine (60 μM) (A48822, Sigma-Aldrich) [26], followed by quantification by 2-hydroxyethylagagarose colony assay (A4018, Sigma-Aldrich). A colony was defined by blue dye stain as >1 mm.

Statistics

Data were expressed as the mean±SEM. Differences between groups were identified by one-way ANOVA and Dunnet's post hoc test. Statistical comparison between two independent groups was determined by the Student's t test or Mann-Whitney U test. The contingency table for the presence of circulating 4T1 cells was analyzed by Fisher's exact test. A p values <0.05 was considered statistically significant (GraphPad Prism 5).

RFS (recurrence-free survival) was defined as the time between initial breast cancer diagnosis and the date of recurrence confirmed by pathology or image study. OS (overall survival) was calculated from the time of initial breast cancer diagnosis to the date of death or last consultation. The Kaplan-Meier method was used to estimate the cumulative incidence of RFS and OS and log-rank tests were used for comparisons (GraphPad Prism 5).

Results:

Identification of MEGF11 in Recurrent TNBC

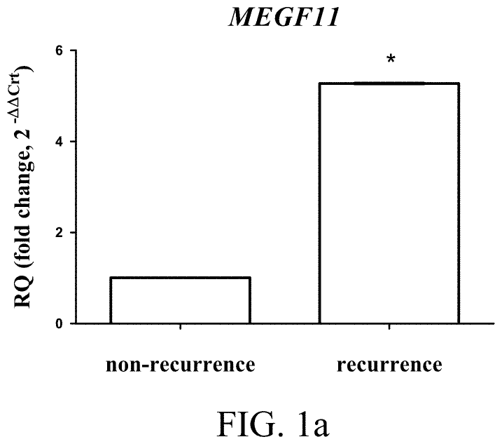

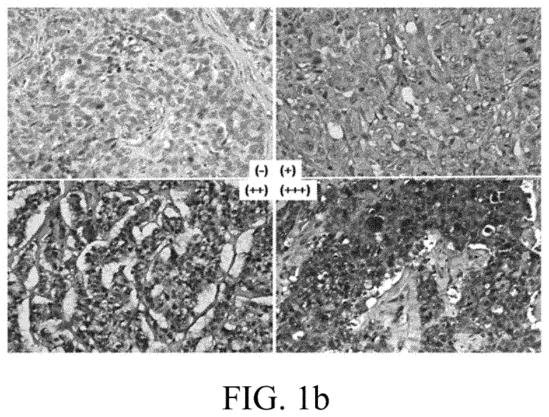

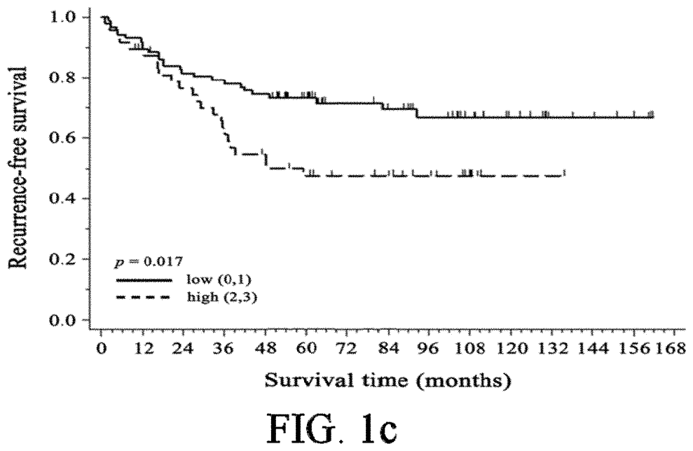

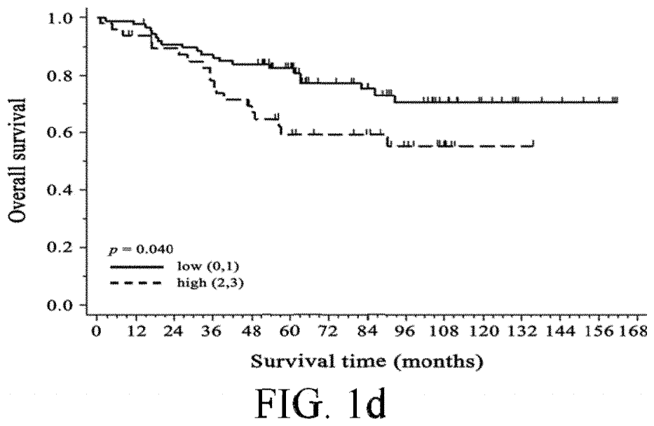

Referring to FIGS. 1a to 1d, these figures show Identification of MEGF11 in recurrent TNBC. To investigate the critical genes related to recurrence in TNBC, the invention conducted cDNA open array analysis on 224 genes in paired TNBC tissue samples (16 recurrent and 24 non-recurrent tissues) and found that MEGF11 was significantly up-regulated in tumor tissues with subsequent clinical recurrence than those without recurrence (as shown in FIG. 1a). Kaplan-Meier plots demonstrated that there was significant negative correlation between MEGF11 protein expression (as shown in FIG. 1b) and RFS (as shown in FIG. 1c) and OS (as shown in FIG. 1d). Besides, results from Kaplan-Meier plotter database spit patient by upper quartile also showed a negative correlation between MEGF11 gene up-regulation and patients' RFS.

Knocked Down MEGF11 in TNBC Cell Lines Decreasing Cell Proliferation Through Suppression of AKT, mTOR and NF-κB-Signaling Pathways

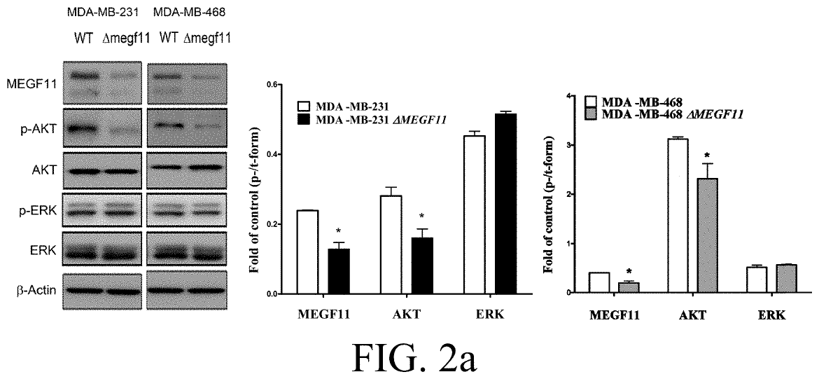

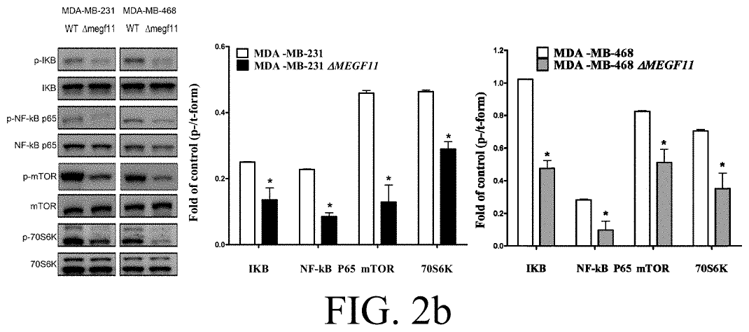

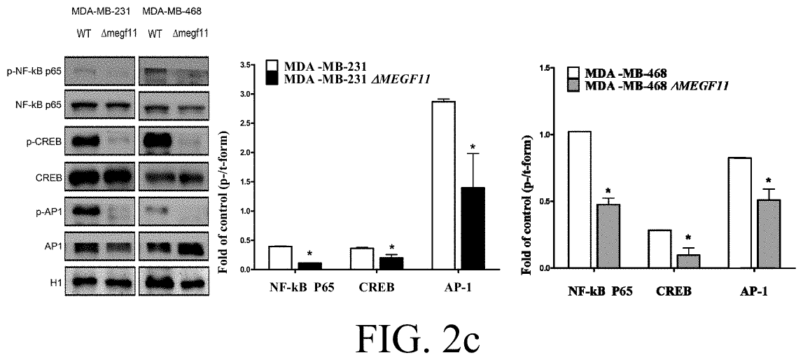

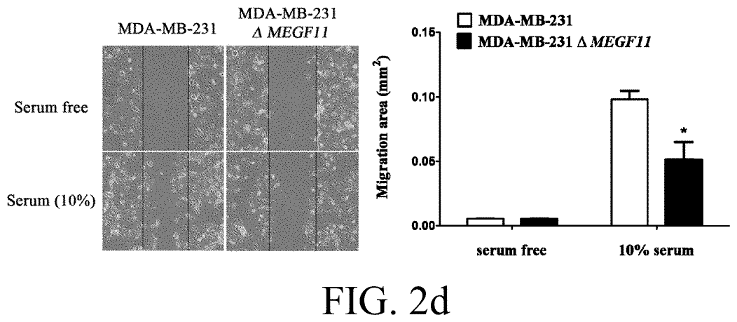

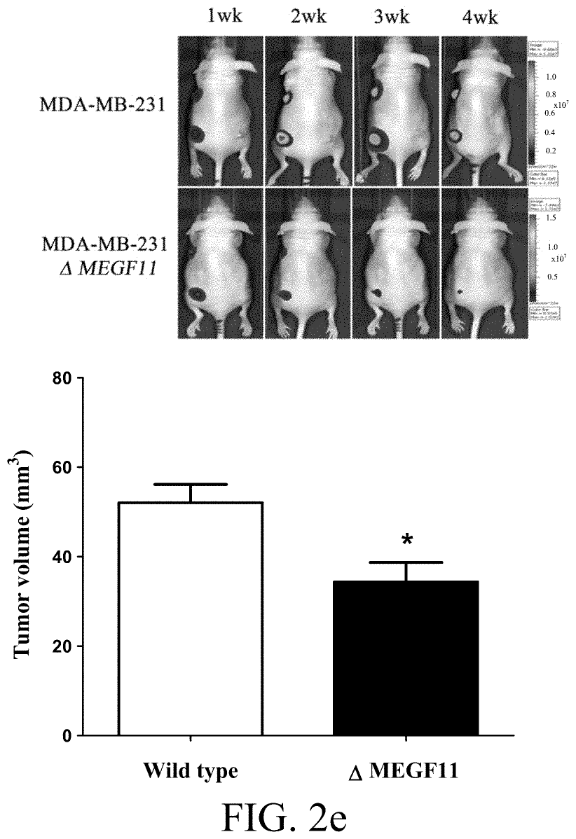

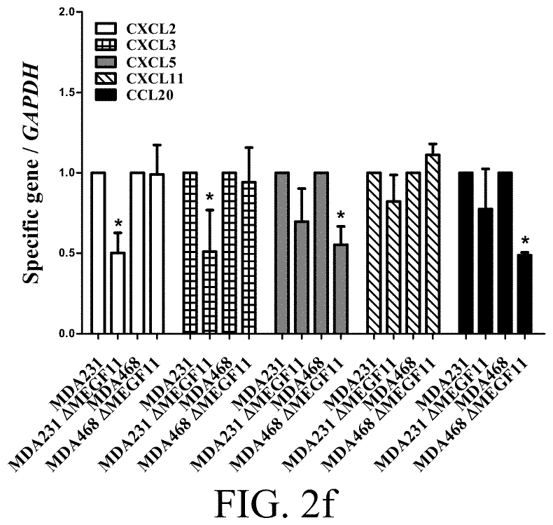

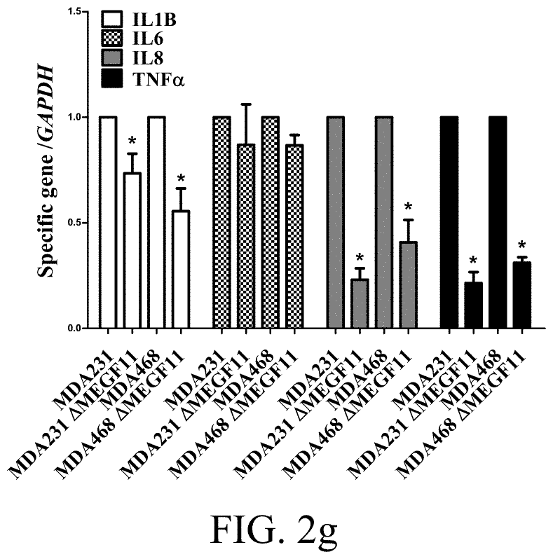

Referring to FIGS. 2a to 2g, these figures show knocked down MEGF11 in TNBC cell lines decreasing cell proliferation through suppression of AKT, mTOR and NF-κB signaling pathways. To determine the roles of MEGF11 in tumor behaviors, the inventors knocked down MEGF11 in TNBC cell lines MDA-MB-231 and MDA-MB-468, and found that—there was significant decreased cell proliferation rate in ΔMEGF11 cells with doubling time in wild type MDA-MB-231/468 cells being 1.57 d and 2.54 d and in ΔMEGF11 MDA-MB-231/468 lines being 4.34 d and 3.25 d, respectively. Western blot analysis disclosed that knock down MEGF11 significantly affect AKT (as shown in FIG. 2a), mTOR and NF-κB signaling (as shown in FIG. 2b) and also decreased the transcription factors such as NF-κB p65, CREB, AP-1 in the nucleus of ΔMEGF11 MDA-MB-231/468 cells (as shown in FIG. 2c). Also, the migration activity (as shown in FIG. 2d) and in vivo growth rate (FIG. 2e) of ΔMEGF11 MDA-MB-231 cells was significantly lower than those of wild type. Of note, many chemokines such as CCL20, CXCL2, CXCL5, and cytokines such as IL1β, TNF-α, IL17-A were down-regulated by knocking down MEGF11 in TNBC cell lines (as shown in FIGS. 2f and 2g). These results suggested that MEGF11 played a role in modulating cell proliferation and cytokines/chemokines production in TNBC cells.

Over-Expression MEGF11 Increasing Up-Regulation of Chemokines, Proinflammatory Cytokines Gene Expression Via AKT Activation, but not Cell Proliferation

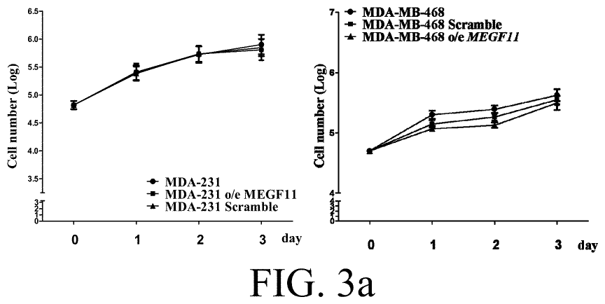

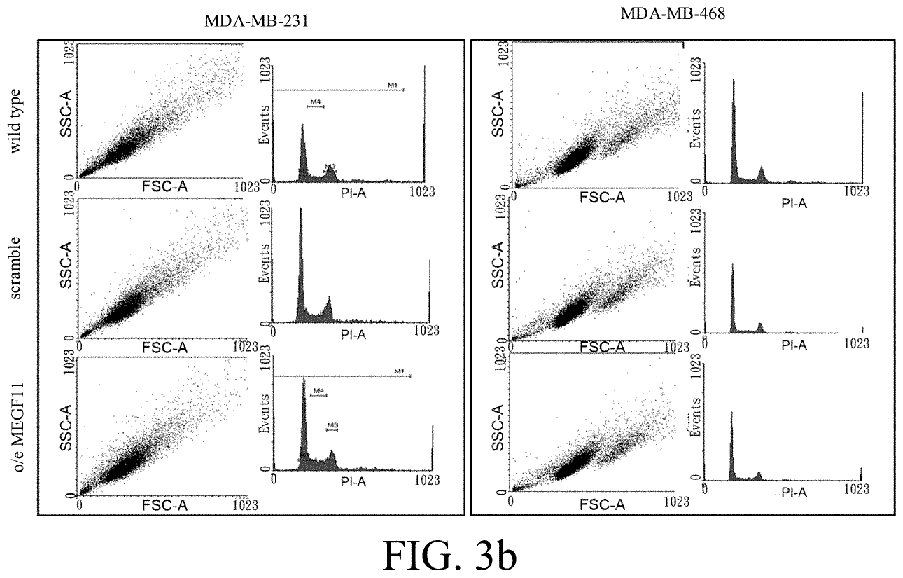

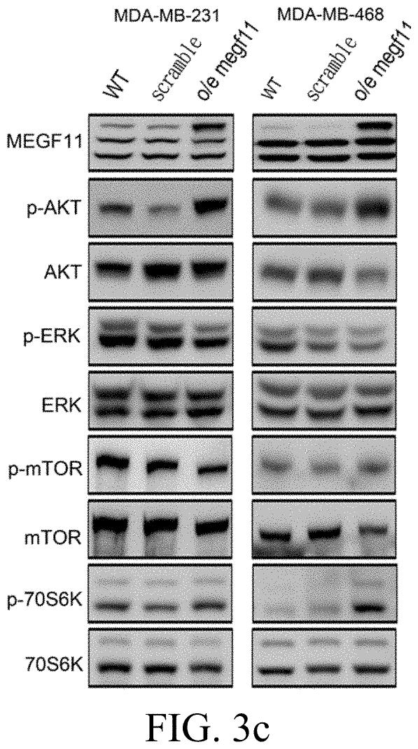

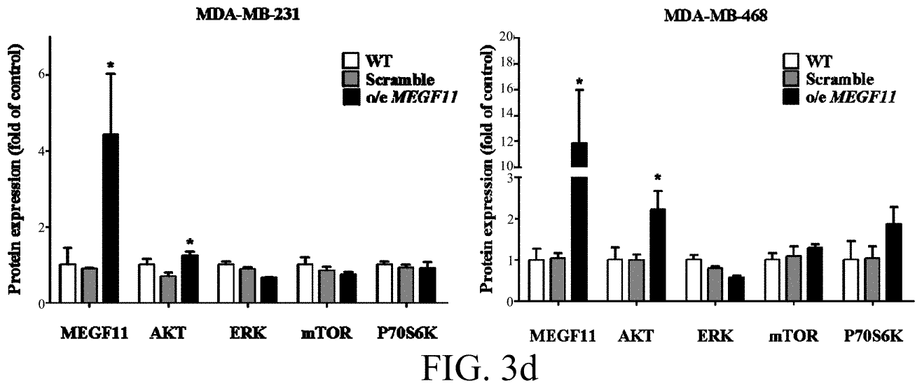

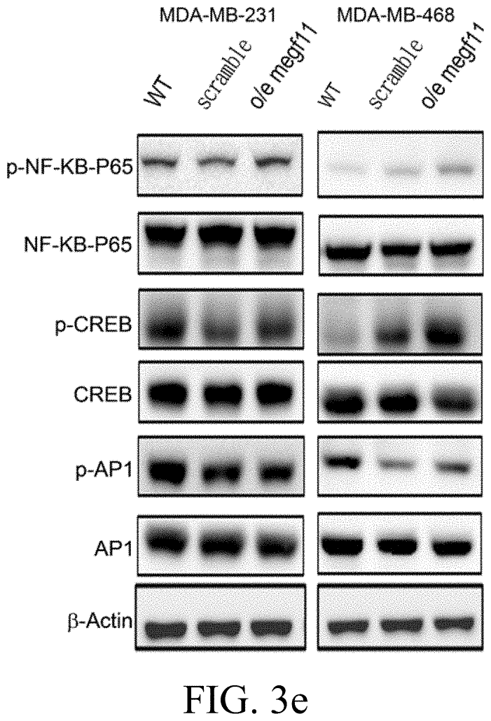

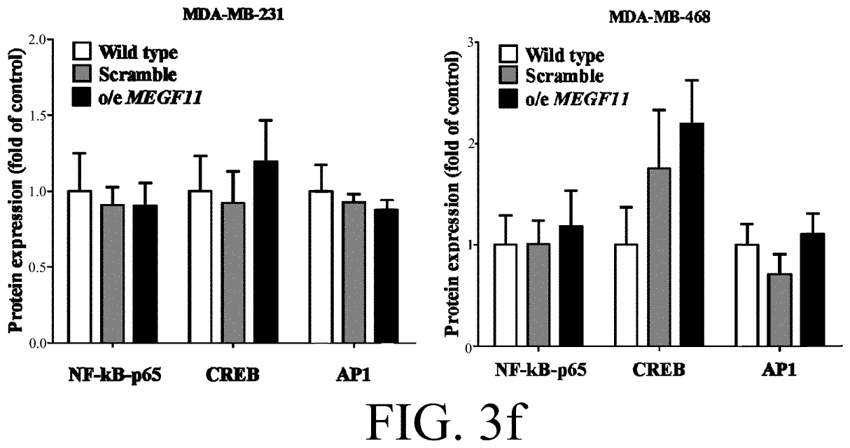

Referring to FIGS. 3a to 3f, these figures show over-expression MEGF11 increasing up-regulation of chemokines, proinflammatory cytokines gene expression via AKT activation, but not cell proliferation. When MEGF11 was over-expressed in TNBC cells, the cell proliferation activity, in terms of cell number (as shown in FIG. 3a), cell cycle analysis (as shown in FIG. 3b), was not increased in MDA-MB-231 and MDA-MB-468 compared to the MEGF11 wild type cells, respectively. When analyzed with Western blot, there was a significantly increased AKT activation, but not ERK, mTOR, p70s6K (as shown in FIGS. 3c and 3d), NF-κB, CREB, and AP-1 activation (as shown in FIGS. 3e and 30 in o/e MEGF11 TNBC cells compared to the scramble groups.

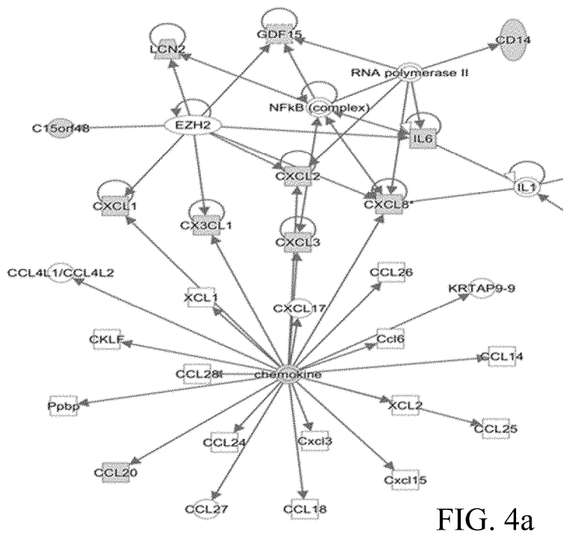

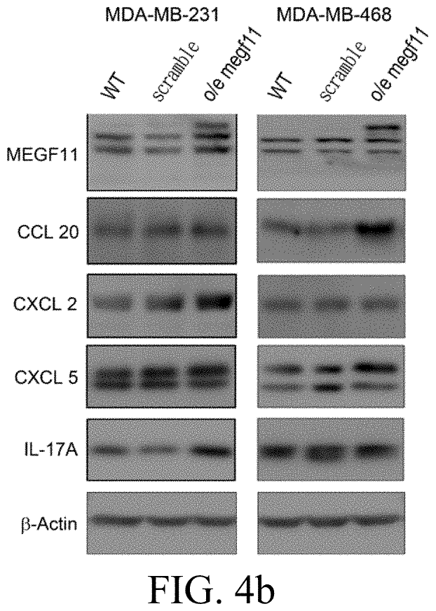

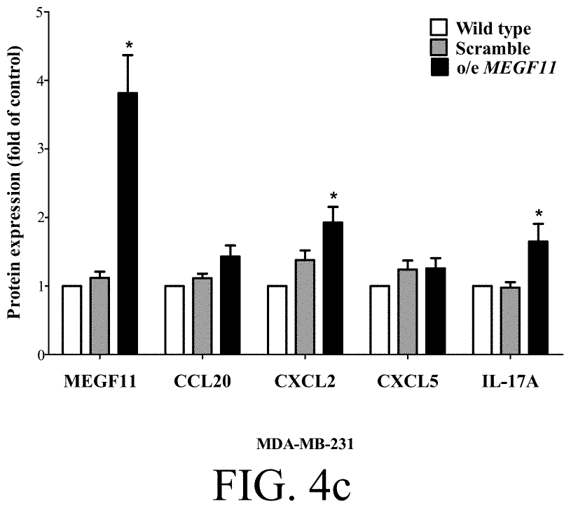

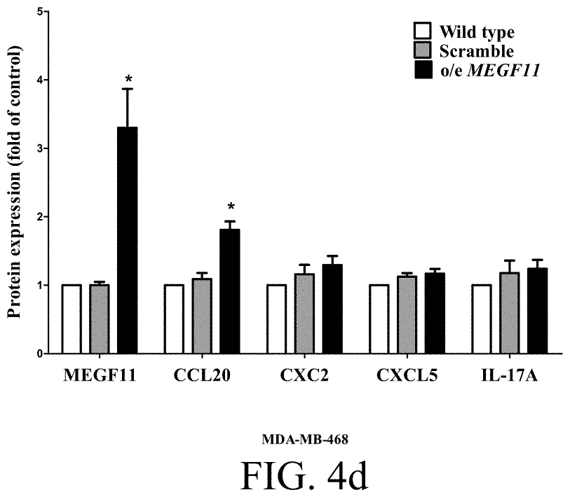

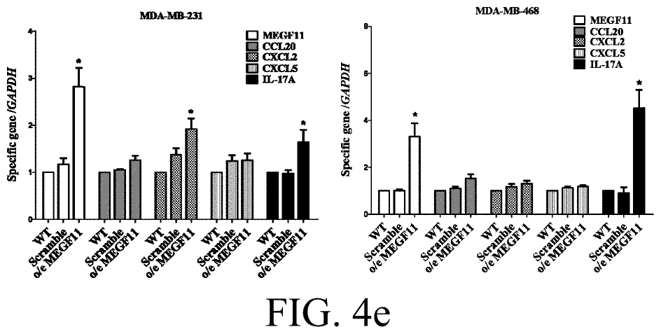

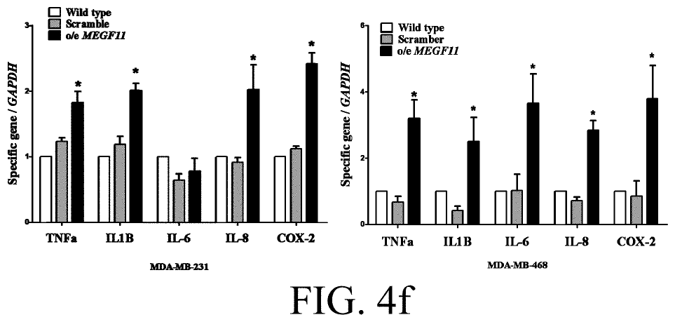

Referring to FIGS. 4a to 4f, these figures show that over-expression of MEGF11 increases up-regulation of chemokines, proinflammatory cytokines gene expression. In contrast, ingenuity pathway analysis disclosed that MEGF11 played important roles in chemokines and cytokines cascades (as shown in FIG. 4a). Western blot (as shown in FIG. 4b) demonstrated that there were increased chemokines such as CCL20, CXCL2 and IL-17A expression in o/e MEGF11 MDA-MB-231 line (as shown in FIG. 4c), but only CCL20 in o/e MDA-MB-468 cells (as shown in FIG. 4d). There were also up-regulated chemokines such as CCL20, CXCL2, CXCL5 genes (as shown in FIG. 4e) and pro-inflammatory cytokines such as TNF-α, IL-1β, and COX2 (as shown in FIG. 40 in over-expressed TNBC cells.

Cross Talk Between MEGF11 and IL-17A

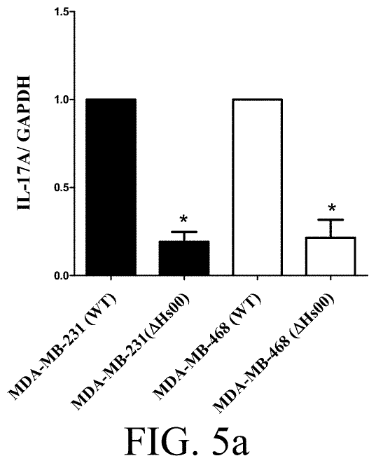

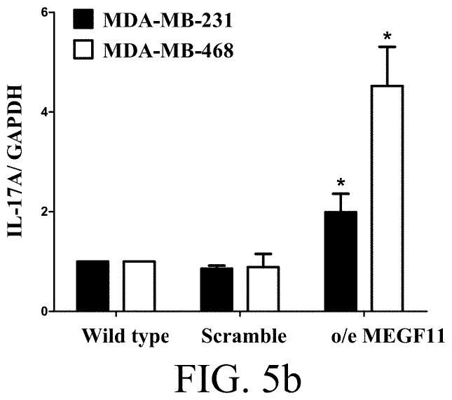

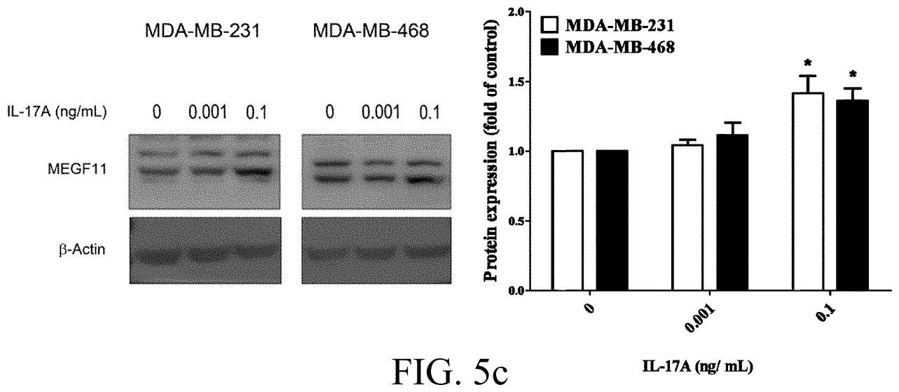

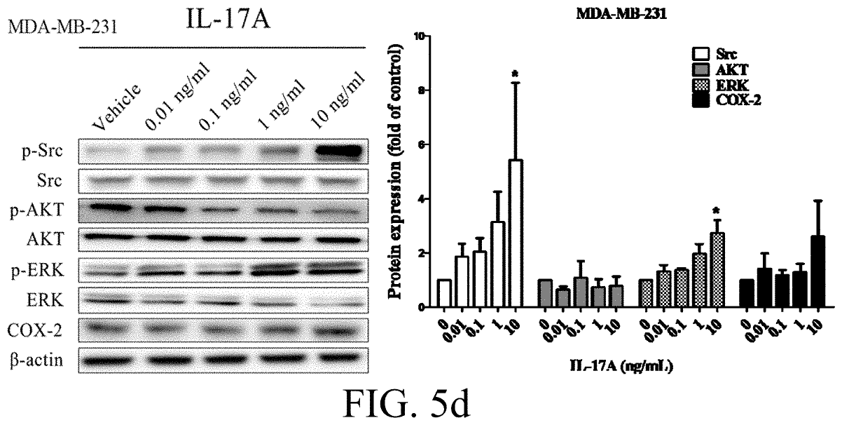

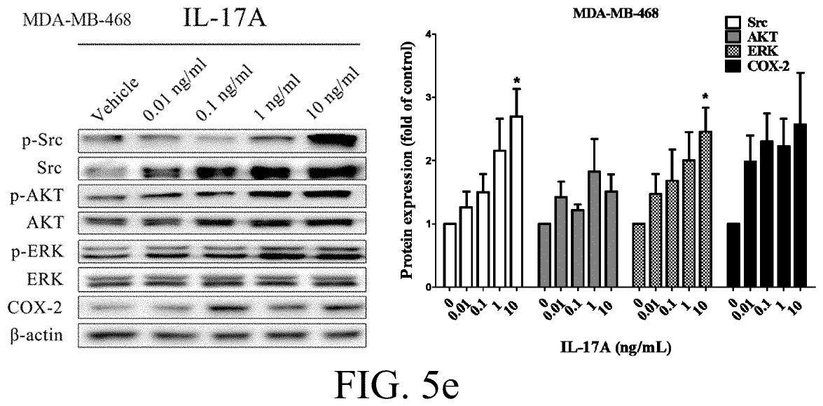

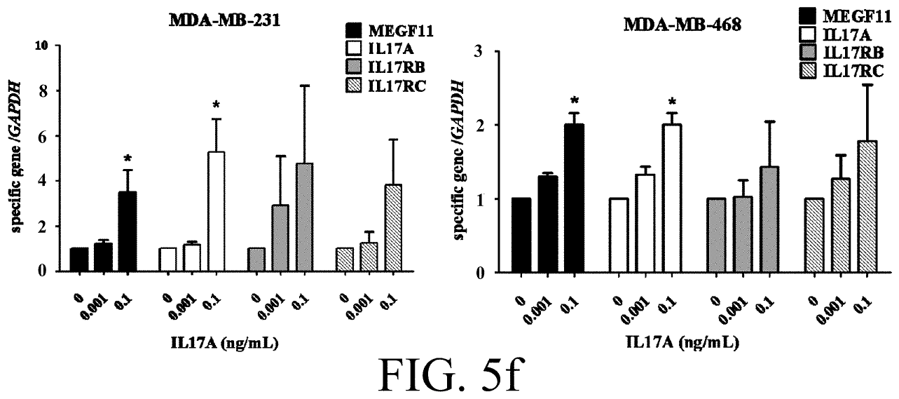

Referring to FIGS. 5a to 5f, these figures show cross talk between MEGF11 and IL-17A. When MEGF11 gene was knocked down in TNBC cell lines, IL-17A transcripts (as shown in FIG. 5a) were decreased in both lines. In contrast, there were increased IL-17A protein expression in MDA-MB-231 (as shown in FIGS. 4b and 4c), but not MDA-MB-468 (as shown in FIGS. 4b and 4d) and increased IL-17A mRNA level in both o/e MEGF11 lines (as shown in FIG. 5b). After administration of IL-17A in cultured media, there was an increased MEGF11 protein (as shown in FIG. 5c), Src and ERK activation (as shown in FIGS. 5d and 5e) and up-regulated MEGF11 and IL-17A genes in MDA-MB-231 and MDA-MB-468 cells (as shown in FIG. 5f). These results indicated a positive cross-talk lineage between MEGF11 and IL-17A and also an IL-17A autocrinal loop in TNBC cells.

IL-17A Increasing Up-Regulation of Chemokines, Proinflammatory Cytokines Gene Expression

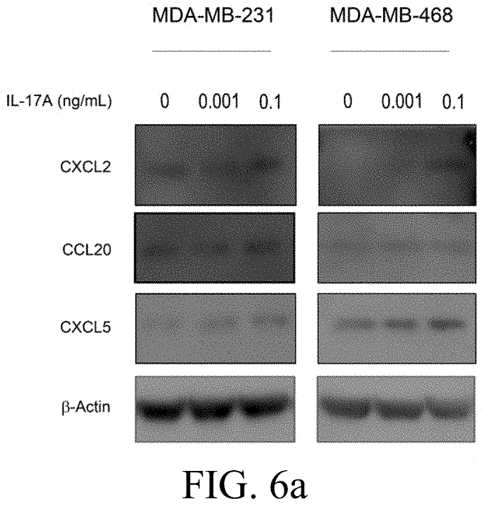

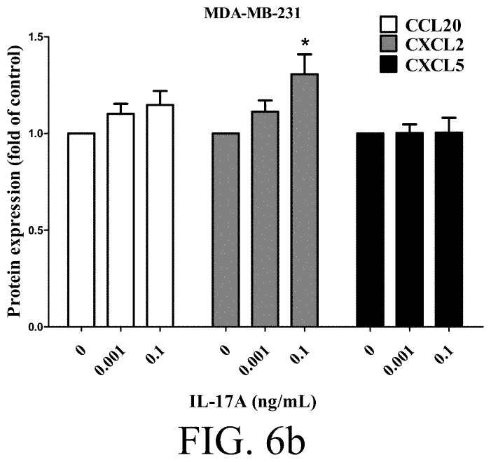

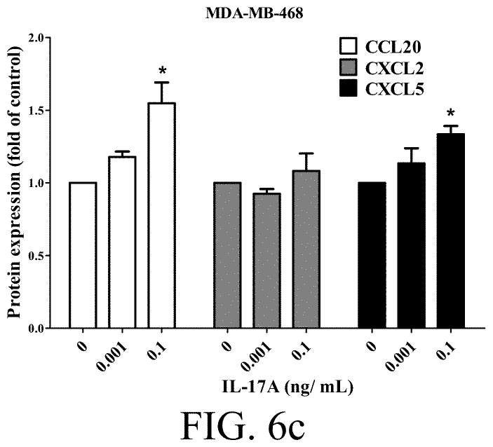

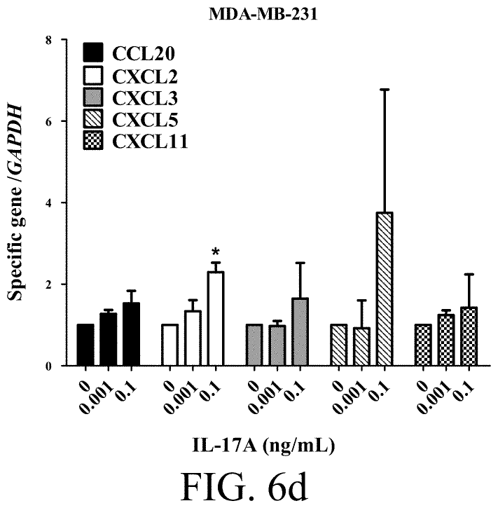

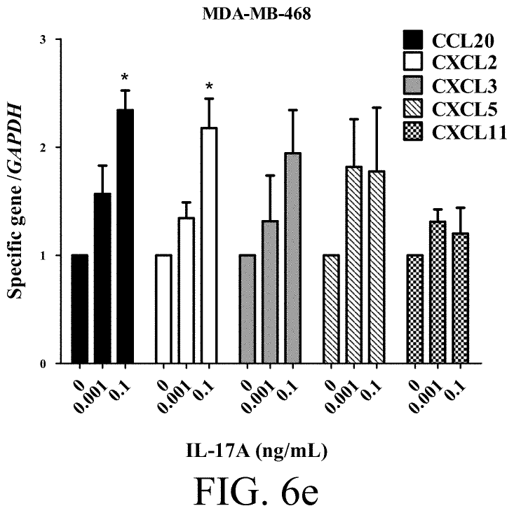

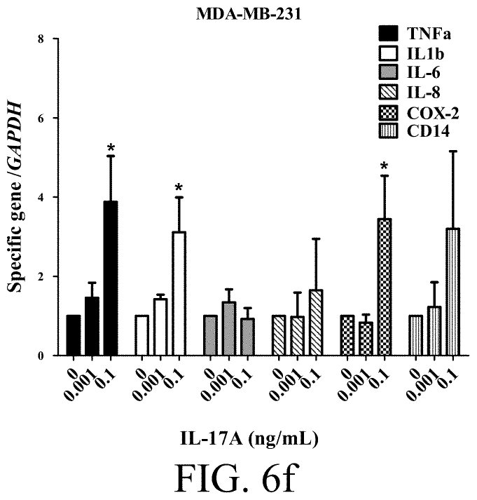

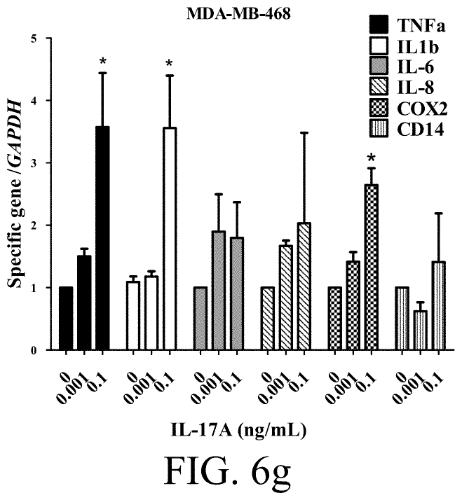

Referring to FIGS. 6a to 6g, these show that IL-17A increases up-regulation of chemokines, proinflammatory cytokines gene expression. Besides increased MEGF11 protein expression, IL-17A significantly increased CXCL2 and CCL20 expression in MDA-MB-231 and MDA-MB-468 line, respectively, both in protein level (FIGS. 6a, 6b and 6c) and mRNA level (FIGS. 6d and 6e). Furthermore, IL-17A up-regulated pro-inflammatory cytokines such as TNFα, IL-1β and COX2 (FIGS. 6f and 6g) in TNBC cells.

Knocked Down MEGF11 in Mouse 4T1 Mammary Cancer Cell Line Decreasing Tumor Weight and Circulating Tumor Cells

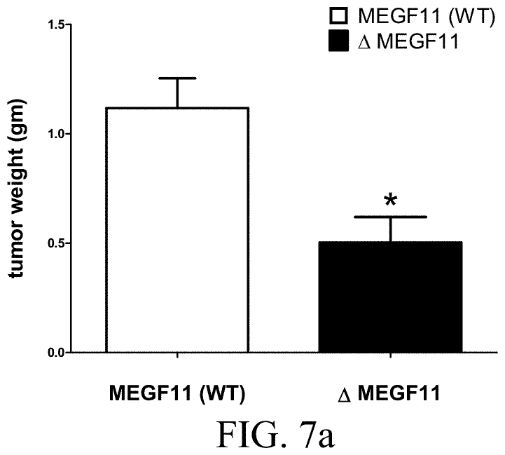

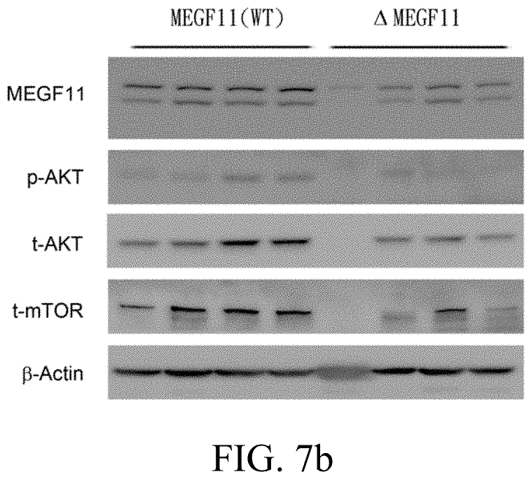

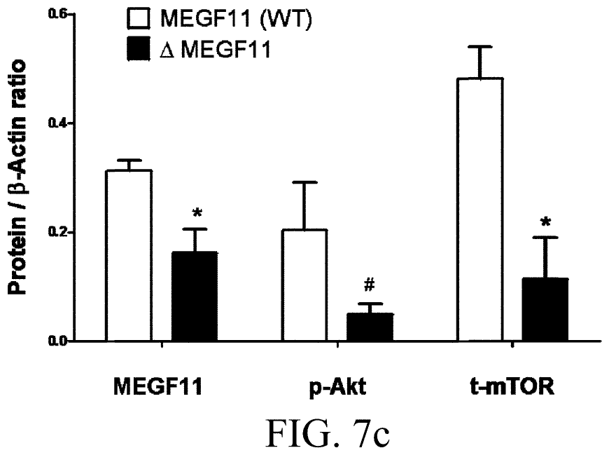

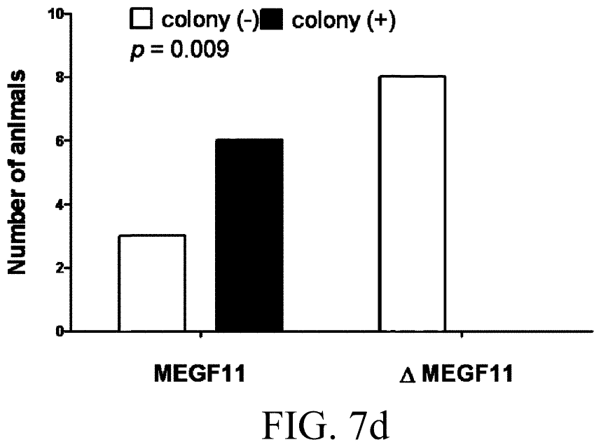

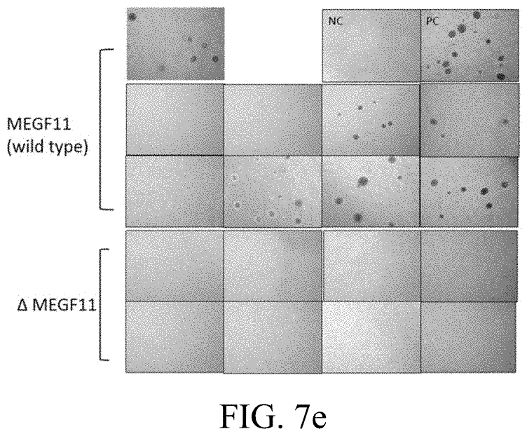

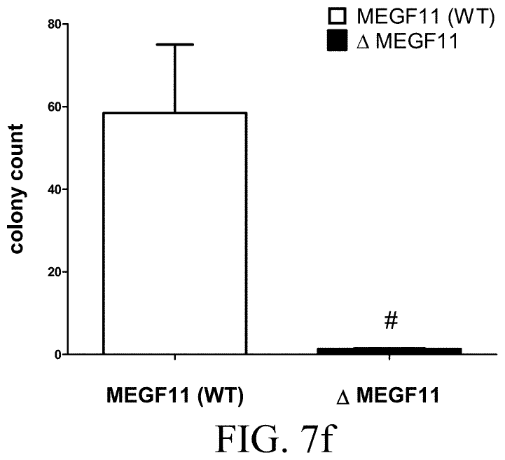

Referring to FIGS. 7a to 7f, these figures show that knocked down MEGF11 in mouse 4T1 mammary cancer cell line decreases tumor weight and circulating tumor cells. Furthermore, using spontaneously occurred mouse mammary tumor 4T1 cell line, the role of MEGF11 was elucidated in mouse metastatic model. After MEGF11 was knocked down in 4T1 cell (ΔMEGF11 4T1), there was a decrease of implanted tumor weight (FIG. 7a) and AKT-mTOR signaling (FIGS. 7b and 7c) compared to MEGF11 wild type. After circulating 4T1 cells were selected with 6-thioguanine, there was a significantly decreased circulating ΔMEGF11 4T1 cells (FIG. 7d) in agar assay (FIGS. 7e and 7f) compared to MEGF11 wild type.

Discussion

Although previous studies suggested that MEGF11 was involved in the formation of mosaics [24] and hematopoietic differentiation [23]. Using cDNA open array analysis for 224 genes on paired TNBC tissue samples (16 recurrent and 24 non-recurrent tissues), the inventors found that MEGF11 was significantly up-regulated in tumor tissues with subsequent clinical recurrence than those without recurrence. In this study, the inventors are the first to demonstrate that the role of MEGF11 in the mechanisms of breast cancer recurrence.

There is evidence that dysregulation of AKT-mTOR signaling, such as AKT overexpression, PI3K amplification/mutation, and loss of PTEN function play an important role in the oncogenesis of many cancers[30], including one subtype of triple negative breast cancer [8, 31]. The inventors' results that knocked down MEGF11 in TNBC cell lines significantly decreased in vitro and in vivo cell proliferation activity via inhibition of AKT, m-TOR and NF-κB signaling, suggesting MEGF11 was essential for the modulation of cell growth. Furthermore, there was no circulating mouse 4T1 cells selected by 6-thioguanine in ΔMEGF11 4T1 line, suggesting MEGF11 plays an important role in tumor metastasis [26]. Of note, our co-localization studies demonstrated that MEGF11 did not co-localized with EGFR or Gs protein on TNBC cells.

Due to the fact that stromal cells and immune cells around tumor microenvironment have been shown to play an important role in predicting the patient's prognosis and the progression of cancer, check point immunotherapy using target monoclonal antibody is involved in the conventional treatments such as chemotherapy or endocrinal therapy for breast cancer [32-34]. The interaction between cancer cells with microenvironment involves not only cell-cell interaction but also the release of many cytokines or chemokines. For example, the presence of TNF-α[35], IL-21[36] and IL-17 have been demonstrated to correlate negatively the patients' prognosis or chemoresistance to paclitaxel [37]. Recent evidence suggests that IL-17A modulates tumor microenvironment by recruitment of immune cells including myeloid-derived suppressor cells (MDSCs), Th17 cells and neutrophils [38, 39]. Interestingly, our results disclose that over-expressed MEGF11 in TNBC cells does not increase cell proliferative activity, but triggers many cytokines and chemokines gene expression leading to cytokine cascades. Furthermore, a positive feedback between MEGF11 and IL-17A in MDA-MB-231/468 is also demonstrated in this study, which might explain the role of MEGF11 in TNBC recurrence.

Recruitment of immune cells is well known to be associated to attenuation of anti-tumor immunity and the increase of anti-therapy effects. In addition to IL-17A, many chemokines are involved in breast cancer progression through a paracrine regulation. For example, breast cancer-derived CXCL1/2 attracted CD11b⁺Gr1⁺ myeloid cells, which promote cell survival and metastasis [40]. Recent evidence suggests that CXCL5 promoted bone metastasis in breast cancer by ERK/MSK1/Elk-1/Snail signaling pathway [41], and CCL20 increase cell proliferation and migration through AKT and MAPK signaling pathways [42]. The inventors' results confirm that up-regulation of MEGF11 significantly increased chemokines expression support the above-mentioned findings.

In addition to immune cells, tumor microenvironment also involves endothelial cells. Our previous study has demonstrated that BDNF promotes migratory activity in tumor cells (MDA-MB-231) and endothelial cells (HUVECs) via autocrine and paracrine regulation, respectively. Besides, overexpression of TrkB, a BDNF receptor, is significantly inversely associated with survival outcome for TNBC patients [43]. The present study shows that over-expressed MEGF11 up-regulates BDNF or TrkB gene expression in TNBC cells, suggesting the role of MEGF11 in tumor cells-endothelial cells interaction.

Given the fact that there is no information concerning the role of MEGF11 in breast cancer, our results demonstrate the MEGF11 is essential for tumor survival and overexpressed MEGF11 induces cytokines and chemokines cascades, which modulate tumor microenvironments in TNBC cells. We conclude that MEGF11 might be a potential therapeutic target for future TNBC treatments.

With the example and explanations above, the features and spirits of the invention will be hopefully well described. Those skilled in the art will readily observe that numerous modifications and alterations of the device may be made while retaining the teaching of the invention. Accordingly, the above disclosure should be construed as limited only by the metes and bounds of the appended claims.

### REFERENCES

[1] Torre L A, Siegel R L, Ward E M, Jemal A. Global Cancer Incidence and Mortality Rates and Trends—An Update. Cancer epidemiology, biomarkers & prevention: a publication of the American Association for Cancer Research, cosponsored by the American Society of Preventive Oncology. 2016; 25:16-27.
[2] Department of Health EY ROCT. 100 Annual Report. 2014.
[3] Podo F, Buydens L M, Degani H, Hilhorst R, Klipp E, Gribbestad I S, et al. Triple-negative breast cancer: present challenges and new perspectives. Molecular oncology. 2010; 4:209-29.
[4] Venkitaraman R. Triple-negative/basal-like breast cancer: clinical, pathologic and molecular features. Expert Rev Anticancer Ther. 2010; 10:199-207.
[5] Thike A A, Cheok P Y, Jara-Lazaro A R, Tan B, Tan P, Tan P H. Triple-negative breast cancer: clinicopathological characteristics and relationship with basal-like breast cancer. Modern pathology: an official journal of the United States and Canadian Academy of Pathology, Inc. 2010; 23:123-33.
[6] Zhang L, Fang C, Xu X, Li A, Cai Q, Long X. Androgen receptor, EGFR, and BRCA1 as biomarkers in triple-negative breast cancer: a meta-analysis. BioMed research international. 2015; 2015:357485.
[7] Lehmann B D, Bauer J A, Chen X, Sanders M E, Chakravarthy A B, Shyr Y, et al. Identification of human triple-negative breast cancer subtypes and preclinical models for selection of targeted therapies. The Journal of clinical investigation. 2011; 121:2750-67.
[8] Lehmann B D, Jovanovic B, Chen X, Estrada M V, Johnson K N, Shyr Y, et al. Refinement of Triple-Negative Breast Cancer Molecular Subtypes: Implications for Neoadjuvant Chemotherapy Selection. PloS one. 2016; 11:e0157368.
[9] Tseng L M, Chiu J H, Liu C Y, Tsai Y F, Wang Y L, Yang C W, et al. A comparison of the molecular subtypes of triple-negative breast cancer among non-Asian and Taiwanese women. Breast Cancer Res Treat. 2017; 163:241-54.
[10] Burstein H J, Elias A D, Rugo H S, Cobleigh M A, Wolff A C, Eisenberg P D, et al. Phase II study of sunitinib malate, an oral multitargeted tyrosine kinase inhibitor, in patients with metastatic breast cancer previously treated with an anthracycline and a taxane. Journal of clinical oncology: official journal of the American Society of Clinical Oncology. 2008; 26:1810-6.
[11] Downing A K, Knott V, Werner J M, Cardy C M, Campbell I D, Handford P A. Solution structure of a pair of calcium-binding epidermal growth factor-like domains: implications for the Marfan syndrome and other genetic disorders. Cell. 1996; 85:597-605.
[12] Bersch B, Hernandez J F, Marion D, Arlaud G J. Solution structure of the epidermal growth factor (EGF)-like module of human complement protease C1r, an atypical member of the EGF family. Biochemistry. 1998; 37:1204-14.
[13] Circolo A, Garnier G, Volanakis J E. A novel murine complement-related gene encoding a C1r-like serum protein. Mol Immunol. 2003; 39:899-906.
[14] Park S Y, Kim S Y, Jung M Y, Bae D J, Kim I S. Epidermal growth factor-like domain repeat of stabilin-2 recognizes phosphatidylserine during cell corpse clearance. Mol Cell Biol. 2008; 28:5288-98.
[15] Pan T C, Sasaki T, Zhang R Z, Fassler R, Timpl R, Chu M L. Structure and expression of fibulin-2, a novel extracellular matrix protein with multiple EGF-like repeats and consensus motifs for calcium binding. J Cell Biol. 1993; 123:1269-77.
[16] Stenflo J, Stenberg Y, Muranyi A. Calcium-binding EGF-like modules in coagulation proteinases: function of the calcium ion in module interactions. Biochim Biophys Acta. 2000; 1477:51-63.
[17] Charlet J, Tomari A, Dallosso A R, Szemes M, Kaselova M, Curry T J, et al. Genome-wide DNA methylation analysis identifies MEGF10 as a novel epigenetically repressed candidate tumor suppressor gene in neuroblastoma. Mol Carcinog. 2017; 56:1290-301.
[18] Mitsuhashi S, Mitsuhashi H, Alexander M S, Sugimoto H, Kang P B. Cysteine mutations cause defective tyrosine phosphorylation in MEGF10 myopathy. FEBS Lett. 2013; 587:2952-7.
[19] Takayama K, Mitsuhashi S, Shin J Y, Tanaka R, Fujii T, Tsuburaya R, et al. Japanese multiple epidermal growth factor 10 (MEGF10) myopathy with novel mutations: A phenotype-genotype correlation. Neuromuscul Disord. 2016; 26:604-9.
[20] Pierson T M, Markello T, Accardi J, Wolfe L, Adams D,
Sincan M, et al. Novel SNP array analysis and exome sequencing detect a homozygous exon 7 deletion of MEGF10 causing early onset myopathy, areflexia, respiratory distress and dysphagia (EMARDD). Neuromuscul Disord. 2013; 23:483-8.
[21] Twigg S R, Lloyd D, Jenkins D, Elcioglu N E, Cooper C D, Al-Sannaa N, et al. Mutations in multidomain protein MEGF8 identify a Carpenter syndrome subtype associated with defective lateralization. Am J Hum Genet. 2012; 91:897-905.
[22] Draper I, Mahoney L J, Mitsuhashi S, Pacak C A, Salomon R N, Kang P B. Silencing of drpr leads to muscle and brain degeneration in adult Drosophila. Am J Pathol. 2014; 184:2653-61.
[23] Krivtsov A V, Rozov F N, Zinovyeva M V, Hendrikx P J, Jiang Y, Visser J W, et al. Jedi—a novel transmembrane protein expressed in early hematopoietic cells. Journal of cellular biochemistry. 2007; 101:767-84.
[24] Kay J N, Chu M W, Sanes J R. MEGF10 and MEGF11 mediate homotypic interactions required for mosaic spacing of retinal neurons. Nature. 2012; 483:465-9.
[25] Tsai C H, Chiu J H, Yang C W, Wang J Y, Tsai Y F, Tseng L M, et al. Molecular characteristics of recurrent triple-negative breast cancer. Molecular medicine reports. 2015; 12:7326-34.
[26] Pulaski B A, Ostrand-Rosenberg S. Mouse 4T1 breast tumor model. Curr Protoc Immunol. 2001; Chapter 20: Unit 20 2.
[27] Tsai C H, Yang C W, Wang J Y, Tsai Y F, Tseng L M, King K L, et al. Timosaponin AIII Suppresses Hepatocyte Growth Factor-Induced Invasive Activity through Sustained ERK Activation in Breast Cancer MDA-MB-231 Cells. Evid Based Complement Alternat Med. 2013; 2013:421051.
[28] Bradford M M. A rapid and sensitive method for the quantitation of microgram quantities of protein utilizing the principle of protein-dye binding. Analytical biochemistry. 1976; 72:248-54.
[29] Chomczynski P, Sacchi N. Single-step method of RNA isolation by acid guanidinium thiocyanate-phenol-chloroform extraction. Analytical biochemistry. 1987; 162: 156-9.
[30] Huang S, Houghton P J. Targeting mTOR signaling for cancer therapy. Curr Opin Pharmacol. 2003; 3:371-7.
[31] Lehmann B D, Pietenpol J A, Tan A R. Triple-negative breast cancer: molecular subtypes and new targets for therapy. American Society of Clinical Oncology educational book American Society of Clinical Oncology Meeting. 2015:e31-9.
[32] Katz H, Alsharedi M. Immunotherapy in triple-negative breast cancer. Medical oncology. 2017; 35:13.
[33] Stovgaard E S, Nielsen D, Hogdall E, Balslev E. Triple negative breast cancer—prognostic role of immune-related factors: a systematic review. Acta Oncol. 2018; 57:74-82.
[34] Solinas C, Gombos A, Latifyan S, Piccart-Gebhart M, Kok M, Buisseret L. Targeting immune checkpoints in breast cancer: an update of early results. ESMO Open. 2017; 2:e000255.
[35] Eftekhari R, Esmaeili R, Mirzaei R, Bidad K, de Lima S, Ajami M, et al. Study of the tumor microenvironment during breast cancer progression. Cancer cell international. 2017; 17:123.
[36] Barjon C, Michaud H A, Fages A, Dejou C, Zampieri A, They L, et al. IL-21 promotes the development of a CD73-positive Vgamma9Vdelta2 T cell regulatory population. Oncoimmunology. 2017; 7:e1379642.
[37] Laprevotte E, Cochaud S, du Manoir S, Lapierre M, Dejou C, Philippe M, et al. The IL-17B-IL-17 receptor B pathway promotes resistance to paclitaxel in breast tumors through activation of the ERK1/2 pathway. Oncotarget. 2017; 8:113360-72.
[38] He D, Li H, Yusuf N, Elmets C A, Li J, Mountz J D, et al. IL-17 promotes tumor development through the induction of tumor promoting microenvironments at tumor sites and myeloid-derived suppressor cells. Journal of immunology (Baltimore, Md.: 1950). 2010; 184:2281-8.
[39] Akbay E A, Koyama S, Liu Y, Dries R, Bufe L E, Silkes M, et al. Interleukin-17A Promotes Lung Tumor Progression through Neutrophil Attraction to Tumor Sites and Mediating Resistance to PD-1 Blockade. J Thorac Oncol. 2017; 12:1268-79.
[40] Acharyya S, Oskarsson T, Vanharanta S, Malladi S, Kim J, Morris P G, et al. A CXCL1 paracrine network links cancer chemoresistance and metastasis. Cell. 2012; 150:165-78.
[41] Hsu Y L, Hou M F, Kuo P L, Huang Y F, Tsai E M. Breast tumor-associated osteoblast-derived CXCL5 increases cancer progression by ERK/MSK1/Elk-1/snail signaling pathway. Oncogene. 2013; 32:4436-47.
[42] Marsigliante S, Vetrugno C, Muscella A. CCL20 induces migration and proliferation on breast epithelial cells. Journal of cellular physiology. 2013; 228:1873-83.
[43] Tsai Y F, Tseng L M, Hsu C Y, Yang M H, Chiu J H, Shyr Y M. Brain-derived neurotrophic factor (BDNF)-TrKB signaling modulates cancer-endothelial cells interaction and affects the outcomes of triple negative breast cancer. PLoS One. 2017; 12:e0178173.

What is claimed is:

1. A method for diagnosing recurrence and treating a triple negative breast cancer (TNBC) in a subject, comprising:
(a) obtaining a first sample from the subject and a second sample from a control culture;
(b) identifying a first relative protein amount of multiple epidermal growth factor-like domains 11 (MEGF11) in the first sample and a second relative protein amount of MEGF11 in the second sample by use of using a PCR-based way;
(c) comparing the first relative protein amount with the second relative protein amount;
(d) if the comparing result in step (b) indicates that the treated subject has an expression of MEGF11 greater than that of the control culture, diagnosing the subject as being in a risk of recurrence of the TNBC; and
(e) administering the subject an effective amount of a composition comprising a shRNA that knocks down MEGF11 expression.

2. The method of claim 1, wherein the control culture comprises non-recurrent tissues, non-recurrent cells and non-recurrent bloods of the TNBC.

3. The method of claim 2, wherein the PCR-based way comprises one or more of a RT-PCR and a real-time PCR.

4. A method for diagnosing recurrence and treating a triple negative breast cancer (TNBC) in a subject, comprising:
(a) obtaining a sample from the subject;
(b) identifying a protein expression of multiple epidermal growth factor-like domains 11 (MEGF11) in the sample by use of using a PCR-based way;
(c) semi-quantifying the protein expression of MEGF11 in the sample;
(d) expressing the semi-quantified the protein expression of MEGF11 in the sample as being a determined value;
(e) judging if the determined value is equal to or greater than a threshold;
(f) if the result in step (e) is YES, diagnosing the subject as being in a risk of recurrence of the TNBC; and
(f) administering the subject an effective amount of a composition comprising a shRNA that knocks down MEGF11 expression.

5. The method of claim 4, wherein the PCR-based way comprises one or more of a RT-PCR and a real-time PCR.

6. The method of claim 5, wherein the threshold is in a range of from 50% to 60%.


SEQUENCE LISTING (157 sequences)

SEQ ID NO: 1 - DNA, 20 bp, Homo sapiens
atggtgctctccctgacggg

SEQ ID NO: 2 - DNA, 20 bp, Homo sapiens
gctcattgccttctccttcc

SEQ ID NO: 3 - DNA, 20 bp, Homo sapiens
tgcaagccacccttgccctg

SEQ ID NO: 4 - DNA, 20 bp, Homo sapiens
aaccccgaggaccccaacgt

SEQ ID NO: 5 - DNA, 20 bp, Homo sapiens
gtgcagccactgggagagct

SEQ ID NO: 6 - DNA, 20 bp, Homo sapiens
atgctgtgactgtccaggaa

SEQ ID NO: 7 - DNA, 20 bp, Homo sapiens
tcgtatgcacaccccttcga

SEQ ID NO: 8 - DNA, 20 bp, Homo sapiens
tcagatctattacacacgat

SEQ ID NO: 9 - DNA, 20 bp, Homo sapiens
gcacagacatcctcaactgg

SEQ ID NO: 10 - DNA, 20 bp, Homo sapiens
ttcaagtgcaccaggcaccg

SEQ ID NO: 11 - DNA, 20 bp, Homo sapiens
gatcagttataagacggcgt

SEQ ID NO: 12 - DNA, 20 bp, Homo sapiens
atcggagaggcctccggacc

SEQ ID NO: 13 - DNA, 20 bp, Homo sapiens
atgtaccggcggaggtccca

SEQ ID NO: 14 - DNA, 20 bp, Homo sapiens
gtgctgccctggctactatg

SEQ ID NO: 15 - DNA, 20 bp, Homo sapiens
agagcggagacttctgcata

SEQ ID NO: 16 - DNA, 20 bp, Homo sapiens
cccctgtgtacggaggagtg

SEQ ID NO: 17 - DNA, 20 bp, Homo sapiens
tgtgcacggccgctgcgttt

SEQ ID NO: 18 - DNA, 20 bp, Homo sapiens
ccccggacacctgccactgc

SEQ ID NO: 19 - DNA, 20 bp, Homo sapiens
gagcctggctggggagggcc

SEQ ID NO: 20 - DNA, 20 bp, Homo sapiens
cgactgctccagcggctgcg

SEQ ID NO: 21 - DNA, 20 bp, Homo sapiens
acagcgaccactgggggccc

SEQ ID NO: 22 - DNA, 20 bp, Homo sapiens
cactgcagcaaccggtgcca

SEQ ID NO: 23 - DNA, 20 bp, Homo sapiens
gtgccagaacggcgccctgt

SEQ ID NO: 24 - DNA, 20 bp, Homo sapiens
gtaaccccatcacaggcgcc

SEQ ID NO: 25 - DNA, 20 bp, Homo sapiens
tgcgtgtgcgccgccggctt

SEQ ID NO: 26 - DNA, 20 bp, Homo sapiens
ccgtggatggcgctgcgagg

SEQ ID NO: 27 - DNA, 20 bp, Homo sapiens
agctctgcgcgcctggcacc

SEQ ID NO: 28 - DNA, 20 bp, Homo sapiens
cacggcaagggatgccagct

SEQ ID NO: 29 - DNA, 20 bp, Homo sapiens
gccgtgccagtgccgacacg

SEQ ID NO: 30 - DNA, 20 bp, Homo sapiens
gtgccagctgcgacccccgc

SEQ ID NO: 31 - DNA, 20 bp, Homo sapiens
gccggcgagtgcctctgcgc

SEQ ID NO: 32 - DNA, 20 bp, Homo sapiens
acctggctacaccggcgtct

SEQ ID NO: 33 - DNA, 20 bp, Homo sapiens
actgcgaggagctgtgccct

SEQ ID NO: 34 - DNA, 20 bp, Homo sapiens
cctgggagccatggagctca

SEQ ID NO: 35 - DNA, 20 bp, Homo sapiens
ctgtgagctgcgctgcccct

SEQ ID NO: 36 - DNA, 20 bp, Homo sapiens
gtcagaatgggggcacctgc

SEQ ID NO: 37 - DNA, 20 bp, Homo sapiens
caccacatcactggcgagtg

SEQ ID NO: 38 - DNA, 20 bp, Homo sapiens
tgcctgccccccaggctgga

SEQ ID NO: 39 - DNA, 20 bp, Homo sapiens
cgggagcagtgtgtgcccag

SEQ ID NO: 40 - DNA, 20 bp, Homo sapiens
ccctgcccaccagggacatt

SEQ ID NO: 41 - DNA, 20 bp, Homo sapiens
tggccagaactgcagccagg

SEQ ID NO: 42 - DNA, 20 bp, Homo sapiens
attgtccttgccaccatgga

SEQ ID NO: 43 - DNA, 20 bp, Homo sapiens
gggcagtgtgaccacgtgac

SEQ ID NO: 44 - DNA, 20 bp, Homo sapiens
tggacagtgccactgtacag

SEQ ID NO: 45 - DNA, 20 bp, Homo sapiens
ctggatacatgggggacagg

SEQ ID NO: 46 - DNA, 20 bp, Homo sapiens
tgccaagaggagtgcccctt

SEQ ID NO: 47 - DNA, 20 bp, Homo sapiens
cgggtccttcggcttccagt

SEQ ID NO: 48 - DNA, 20 bp, Homo sapiens
gctcacagcgctgtgactgc

SEQ ID NO: 49 - DNA, 20 bp, Homo sapiens
cacaatggggggcagtgttc

SEQ ID NO: 50 - DNA, 20 bp, Homo sapiens
acccaccacgggtgcctgcg

SEQ ID NO: 51 - DNA, 20 bp, Homo sapiens
agtgtgagcctggctacaag

SEQ ID NO: 52 - DNA, 20 bp, Homo sapiens
ggcccacgctgccaggagcg

SEQ ID NO: 53 - DNA, 20 bp, Homo sapiens
actgtgcccggagggcctgc

SEQ ID NO: 54 - DNA, 20 bp, Homo sapiens
atggcccaggctgcaccctg

SEQ ID NO: 55 - DNA, 20 bp, Homo sapiens
ccctgcccctgtgacgctga

SEQ ID NO: 56 - DNA, 20 bp, Homo sapiens
caacaccatcagctgccacc

SEQ ID NO: 57 - DNA, 20 bp, Homo sapiens
cagtaactggagcttgtacc

SEQ ID NO: 58 - DNA, 20 bp, Homo sapiens
tgccagccaggctggtctgg

SEQ ID NO: 59 - DNA, 20 bp, Homo sapiens
tcaccactgcaatgaatcct

SEQ ID NO: 60 - DNA, 20 bp, Homo sapiens
gccctgttggctactatggc

SEQ ID NO: 61 - DNA, 20 bp, Homo sapiens
gatggctgccagctgccttg

SEQ ID NO: 62 - DNA, 20 bp, Homo sapiens
cacctgtcagaatggcgccg

SEQ ID NO: 63 - DNA, 20 bp, Homo sapiens
actgccacagcatcactggg

SEQ ID NO: 64 - DNA, 20 bp, Homo sapiens
ggctgcacttgtgctccggg

SEQ ID NO: 65 - DNA, 20 bp, Homo sapiens
cttcatgggagaggtctgtg

SEQ ID NO: 66 - DNA, 20 bp, Homo sapiens
ccgtttcctgtgcagcaggg

SEQ ID NO: 67 - DNA, 20 bp, Homo sapiens
acctatggccccaactgctc

SEQ ID NO: 68 - DNA, 20 bp, Homo sapiens
gtccatctgtagctgtaaca

SEQ ID NO: 69 - DNA, 20 bp, Homo sapiens
atggtggcacctgctcccca

SEQ ID NO: 70 - DNA, 20 bp, Homo sapiens
gtagatggctcctgtacctg

SEQ ID NO: 71 - DNA, 20 bp, Homo sapiens
caaggaagggtggcagggcc

SEQ ID NO: 72 - DNA, 20 bp, Homo sapiens
tggactgcaccctgccatgt

SEQ ID NO: 73 - DNA, 20 bp, Homo sapiens
cccagtgggacgtggggcct

SEQ ID NO: 74 - DNA, 20 bp, Homo sapiens
gaactgcaacgagagctgca

SEQ ID NO: 75 - DNA, 20 bp, Homo sapiens
cctgtgccaatggggcagcc

SEQ ID NO: 76 - DNA, 20 bp, Homo sapiens
tgcagccccatagacggctc

SEQ ID NO: 77 - DNA, 20 bp, Homo sapiens
ctgctcctgcactcctggct

SEQ ID NO: 78 - DNA, 20 bp, Homo sapiens
ggctgggagacacctgtgag

SEQ ID NO: 79 - DNA, 20 bp, Homo sapiens
ctgccttgcccggatggcac

SEQ ID NO: 80 - DNA, 20 bp, Homo sapiens
atttgggctgaactgcagtg

SEQ ID NO: 81 - DNA, 20 bp, Homo sapiens
aacactgtgactgcagccat

SEQ ID NO: 82 - DNA, 20 bp, Homo sapiens
gctgatggatgtgaccccgt

SEQ ID NO: 83 - DNA, 20 bp, Homo sapiens
cacaggccactgctgctgcc

SEQ ID NO: 84 - DNA, 20 bp, Homo sapiens
tggccggatggacaggcatc

SEQ ID NO: 85 - DNA, 20 bp, Homo sapiens
cgctgtgacagcacgtgtcc

SEQ ID NO: 86 - DNA, 20 bp, Homo sapiens
acctggccgctggggcccca

SEQ ID NO: 87 - DNA, 20 bp, Homo sapiens
actgctctgtctcctgcagc

SEQ ID NO: 88 - DNA, 20 bp, Homo sapiens
tgtgagaatggaggctcctg

SEQ ID NO: 89 - DNA, 20 bp, Homo sapiens
ctccccagaggatgggagct

SEQ ID NO: 90 - DNA, 20 bp, Homo sapiens
gcgagtgtgcccctggcttc

SEQ ID NO: 91 - DNA, 20 bp, Homo sapiens
cgaggacccttatgccagag

SEQ ID NO: 92 - DNA, 20 bp, Homo sapiens
aatctgcccccctgggttct

SEQ ID NO: 93 - DNA, 20 bp, Homo sapiens
atggccacggctgcgcccag

SEQ ID NO: 94 - DNA, 20 bp, Homo sapiens
ccatgccccctctgcgtgca

SEQ ID NO: 95 - DNA, 20 bp, Homo sapiens
cagcagcaggccctgccacc

SEQ ID NO: 96 - DNA, 20 bp, Homo sapiens
acatcagcggcatctgtgag

SEQ ID NO: 97 - DNA, 20 bp, Homo sapiens
tgcctcccaggattctctgg

SEQ ID NO: 98 - DNA, 20 bp, Homo sapiens
agctctctgcaaccaagtgt

SEQ ID NO: 99 - DNA, 20 bp, Homo sapiens
gtgctggaggatactttggg

SEQ ID NO: 100 - DNA, 20 bp, Homo sapiens
caggactgtgcccagctctg

SEQ ID NO: 101 - DNA, 20 bp, Homo sapiens
ctcctgtgccaacaacggga

SEQ ID NO: 102 - DNA, 20 bp, Homo sapiens
cctgcagccctatcgatggc

SEQ ID NO: 103 - DNA, 20 bp, Homo sapiens
tcctgccagtgctttcctgg

SEQ ID NO: 104 - DNA, 20 bp, Homo sapiens
atggattggcaaggactgct

SEQ ID NO: 105 - DNA, 20 bp, Homo sapiens
cacaggcttgcccacccggg

SEQ ID NO: 106 - DNA, 20 bp, Homo sapiens
ttctggggccccgcctgctt

SEQ ID NO: 107 - DNA, 20 bp, Homo sapiens
ccacgcatgcagctgccaca

SEQ ID NO: 108 - DNA, 20 bp, Homo sapiens
acggggcgagctgcagcgcc

SEQ ID NO: 109 - DNA, 20 bp, Homo sapiens
gaggacggggcctgccactg

SEQ ID NO: 110 - DNA, 20 bp, Homo sapiens
cacccctggctggactggac

SEQ ID NO: 111 - DNA, 20 bp, Homo sapiens
tcttctgcacacagcgctgc

SEQ ID NO: 112 - DNA, 20 bp, Homo sapiens
ccagcagcattttttgggaa

SEQ ID NO: 113 - DNA, 20 bp, Homo sapiens
ggactgtgggcgcgtatgcc

SEQ ID NO: 114 - DNA, 20 bp, Homo sapiens
agtgtcagaatggcgccagc

SEQ ID NO: 115 - DNA, 20 bp, Homo sapiens
tgtgaccacatcagtggcaa

SEQ ID NO: 116 - DNA, 20 bp, Homo sapiens
gtgcacctgccgcacaggct

SEQ ID NO: 117 - DNA, 20 bp, Homo sapiens
tcaccgggcaacactgtgag

SEQ ID NO: 118 - DNA, 20 bp, Homo sapiens
cagagatgtgccccaggaac

SEQ ID NO: 119 - DNA, 20 bp, Homo sapiens
ctttggctatgggtgtcagc

SEQ ID NO: 120 - DNA, 20 bp, Homo sapiens
agctatgtgagtgcatgaac

SEQ ID NO: 121 - DNA, 20 bp, Homo sapiens
aactccacctgtgaccatgt

SEQ ID NO: 122 - DNA, 20 bp, Homo sapiens
caccggcacctgttactgca

SEQ ID NO: 123 - DNA, 20 bp, Homo sapiens
gccctggcttcaaaggaatc

SEQ ID NO: 124 - DNA, 20 bp, Homo sapiens
aggtgtgaccaagctgccct

SEQ ID NO: 125 - DNA, 20 bp, Homo sapiens
catgatggaggagctgaatc

SEQ ID NO: 126 - DNA, 20 bp, Homo sapiens
cctacaccaagatcagccca

SEQ ID NO: 127 - DNA, 20 bp, Homo sapiens
gcactgggtgcagagcggca

SEQ ID NO: 128 - DNA, 20 bp, Homo sapiens
ctcggtgggtgctgtcacag

SEQ ID NO: 129 - DNA, 20 bp, Homo sapiens
gcatcatgctcctgttattc

SEQ ID NO: 130 - DNA, 20 bp, Homo sapiens
ctcattgtggtgctgctggg

SEQ ID NO: 131 - DNA, 20 bp, Homo sapiens
cctatttgcctggcatcggc

SEQ ID NO: 132 - DNA, 20 bp, Homo sapiens
ggcggcagaaagagaagggc

SEQ ID NO: 133 - DNA, 20 bp, Homo sapiens
cgagacctggctccccgtgt

SEQ ID NO: 134 - DNA, 20 bp, Homo sapiens
ctcctacacacctgccatga

SEQ ID NO: 135 - DNA, 20 bp, Homo sapiens
ggatgaccagcaccgactac

SEQ ID NO: 136 - DNA, 20 bp, Homo sapiens
tccctctcaggtgcttgtgg

SEQ ID NO: 137 - DNA, 20 bp, Homo sapiens
aatggatagacgtcagaaca

SEQ ID NO: 138 - DNA, 20 bp, Homo sapiens
catacattatggacaaaggc

SEQ ID NO: 139 - DNA, 20 bp, Homo sapiens
ttcaaagattacatgaaaga

SEQ ID NO: 140 - DNA, 20 bp, Homo sapiens
atccgtgtgcagttctagta

SEQ ID NO: 141 - DNA, 20 bp, Homo sapiens
cttgttccttgaatagcagt

SEQ ID NO: 142 - DNA, 20 bp, Homo sapiens
gaaaacccttacgccacaat

SEQ ID NO: 143 - DNA, 20 bp, Homo sapiens
taaggacccacccatcctca

SEQ ID NO: 144 - DNA, 20 bp, Homo sapiens
cctgcaagcttccagaaagc

SEQ ID NO: 145 - DNA, 20 bp, Homo sapiens
agctatgtagaaatgaagtc

SEQ ID NO: 146 - DNA, 20 bp, Homo sapiens
gcctgtgcacatggggtctc

SEQ ID NO: 147 - DNA, 20 bp, Homo sapiens
cgtacacagatgtgccatcc

SEQ ID NO: 148 - DNA, 20 bp, Homo sapiens
ttgtcgacatctaataaaaa

SEQ ID NO: 149 - DNA, 20 bp, Homo sapiens
tatatatgaagttgagccca

SEQ ID NO: 150 - DNA, 20 bp, Homo sapiens
cagtcagtgtggtccaagaa

SEQ ID NO: 151 - DNA, 20 bp, Homo sapiens
ggttgcggtcataactccag

SEQ ID NO: 152 - DNA, 20 bp, Homo sapiens
ctatatccagaatgcatacg

SEQ ID NO: 153 - DNA, 20 bp, Homo sapiens
acctacctaggaacagccat

SEQ ID NO: 154 - DNA, 20 bp, Homo sapiens
attcctggtcattatgacct

SEQ ID NO: 155 - DNA, 20 bp, Homo sapiens
cctcccagtaagacagagcc

SEQ ID NO: 156 - DNA, 20 bp, Homo sapiens
ctgccaatgggccgtcccag

SEQ ID NO: 157 - DNA, 15 bp, Homo sapiens
gacaagcaatcttaa